(19) United States
(12) Patent Application Publication
(10) Pub. No.: US 2015/0335794 A1
(43) Pub. Date: Nov. 26, 2015

(54) SMART COATING FOR IMPLANTABLE DEVICES

(71) Applicant: DEBIOTECH S.A., Lausanne (CH)

(72) Inventors: Laurent-Dominique PIVETEAU, Bussigny (CH); Frédéric NEFTEL, Lausanne (CH)

(73) Assignee: DEBIOTECH S.A., Lausanne (CH)

(21) Appl. No.: 14/649,375

(22) PCT Filed: Dec. 3, 2013
(86) PCT No.: PCT/IB2013/060588

§ 371 Date: Jun. 3, 2015

(30) Foreign Application Priority Data
Dec. 3, 2012 (EP) 12195245.1

(51) Int. Cl. A61L 31/14; A61L 31/16; A61L 31/10; A61L 27/54; A61L 29/08; A61L 27/58; A61L 27/52; A61L 29/14; A61L 29/16; A61F 2/00; A61L 27/34

(52) U.S. Cl. CPC A61L 31/148; A61F 2/0077; A61L 31/145; A61L 31/16; A61L 31/10; A61L 27/54; A61L 27/34; A61L 27/58; A61L 27/52; A61L 29/145; A61L 29/148; A61L 29/16; A61L 29/085; A61F 2002/0086; A61L 2300/604; A61L 2300/416

(57) ABSTRACT

The present invention relates to a coating for medical implants. The coating contains an active agent that is intended to have an effect on the surrounding tissue after placement of the implant. This effect is activated in the presence of cells. The active agent is released from the coating under the direct influence of the cells.

### FIELD OF INVENTION

The present invention relates to a coating for medical implants. In particular it relates to coatings that will react in the presence of cells, and more specifically in the presence of one or several specific cells which may be the targeted cells.

### STATE OF THE ART

The standard therapy used to treat the blockage of a coronary artery includes the placement into the (partially) obstructed artery of a stent, a small metallic mesh which is intended to maintain the vessel open after the intervention. It is positioned into the artery with a metallic guide and, once in place, is propped open thanks to a small balloon. One of the most common issues following this type of intervention is restenosis: a post operatory re-closing of the vessel. This occurs in the weeks following the intervention and is due to the anarchical growth of smooth muscle cells (SMC) induced by the trauma of the procedure. In order to limit this occurrence, a majority of stents today is covered with a drug layer (most often integrated into a polymeric structure) which is released over time and acts as a cytotoxic agent against the SMC, therefore blocking their development. This elution process, that takes a few weeks, is controlled in different ways depending on the stent platform: the drug can be loaded in a reservoir-like structure and be released by diffusion through a porous membrane; it may be embedded into a porous membrane and diffuse through the membrane; it may be dissolved into a polymer that will erode over time; or it may simply be attached at the surface of the stent in which case the elution rate is controlled by its own solubility or by the solubility of the link that attaches it to the substrate.

One of the issues that has been raised with most of these coating is the long term toxicity of the material used to store the drug. Different solutions to this problem have been proposed. In WO 03/090818, the coating containing the drug will dissolve over time leaving a pure bare metal stent in the vessel a few months after implantation. In WO 2010/136848 and WO 2007/148240, which are incorporated herein by reference in their entirety, coatings made of a nanoporous layer containing buried micrometric reservoirs are described. In particular this type of coating is manufactured using ceramics, with the objective of reducing the long term drawbacks of commonly used polymeric materials.

All these systems remain fully passive. Once placed into a solution, the drug present into the coating immediately starts to be released. The elution profile is defined by various physico-chemical parameters such as the concentration of the drug in the coating, its solubility, or the type of matrix containing the molecule. It is never initiated or driven by the real need, which is the presence of the cells to be eliminated or treated.

Other systems are using an intermediary signalling system. In D. Koley et al. “Toward a smart catheter that senses microbial attachment and turns on electrochemical release of bactericidal nitric oxide” ACS Meeting 2012, Philadelphia, Aug. 19-23, 2012, a coating is activated following a change of pH in the environment. This change of pH, which may be induced by the presence of bacteria and the subsequent reaction of the environment, will activate the coating that will release a bacteriostatic agent. This type of device allows a more specific and targeted response than purely passive systems. However it does not offer the level of efficacy and safety proposed in this application. In particular, the change of pH is not specific to the presence of bacteria, or targeted cells and other causes may induce a non-desired release of the drug.

In US 2004/0170685 to Carpenter et a., which is incorporated herein by reference in its entirety, a stent coated with a biodegradable, bioactive coating is described. The polymer of the coating comprises at least one bioactive agent that is covalently bound to the polymer and that produces a therapeutic effect in situ. However, the mechanisms involved in the degradation of the coating are triggered by the environment in a non-specific way. The polymer contains moieties that are hydrolysed leading to its breakage in smaller products. This process starts immediately after the placement of the stent in the artery, independently of the presence of the targeted cells.

In US 2007/0280991 to Glauser et al., which is incorporated herein by reference in its entirety, a stent is presented with a coating which contains both an hydrophilic and an hydrophobic drugs. The release of the hydrophilic drug is controlled by attaching it to a polymer present in the coating. Here again, the release process is completely independent of the presence or the absence of the targeted cells.

In US 2011/0165244 to Neff, which is incorporated herein by reference in its entirety, a material is presented that contains a bioactive agent linked to a copolymer through a labile linker. The copolymer is a mixture of hydrophilic and hydrophobic domains. The labile linker may be selectively cleaved by a physiologic stimulus, separating the bioactive agent from the copolymer.

In US 2008/0146992 to Hossainy et al., which is incorporated herein by reference in its entirety, a coating is presented that comprises a conjugate of a bioactive agent and a copolymer that comprises itself at least one acrylamide monomer. It further describes two ways of releasing the bioactive agent from the matrix: one if the bioactive agent is not covalently bounded to the copolymer with the agent diffusing through the matrix and another one where the agent is covalently bounded to the copolymer with the labile link between the agent and the copolymer is degraded or disrupted.

In WO 02/055122 to Lewis et al., which is incorporated herein by reference in its entirety, a stent is presented that has a polymer coating made of an amphiphilic polymer comprising a sparingly water soluble drug. When placed in an aqueous environment, the polymer will swell, allowing the permeation of the drug.

In US 2005/0238689 to Carpenter et al., which is incorporated herein by reference in its entirety, a bioactive stent coated with a bioactive, biodegradable polymer is presented. The polymer bears covalently linked bioligands that will specifically capture and activate progenitors of endothelial cells from the blood of the patient.

### GENERAL DESCRIPTION OF THE INVENTION

The present application claims the benefit of the priority of the application EP 12195245.1 filed on 3 Dec. 2012 in the name of Debiotech SA, the entire disclosure of which is incorporated herein by reference.

The invention is set forth and characterized in the independent claims, while the dependent claims describe other characteristics of the invention.

The principle of the invention is a coating for an implantable device which comprises an active agent delivered and/or acted when the coating is in contact or in presence of at least one specific cell which may be the targeted cell.

In one embodiment, the invention discloses a coating, for a medical device, which comprises an active agent which is released in a controlled manner. Said coating furthermore comprises a structural material and substance, wherein the substance is adapted to induce the dissolution of the coating in such a way to release the active agent when said substance is in contact with or in presence of a specific cell. Said cell may be in a specific state to induce said dissolution. Said active agent may be adapted to target a cell type and/or act on a cell type.

In one embodiment, the present invention relates to a coating for an implantable medical device comprising a structural material and an active agent. Said structural material and/or active agent contain a substance which, when brought into contact with a cell, induces the dissolution of said structural material and/or of said substance and thereby allows the release of said active agent.

In one embodiment, the present invention relates to a coating for an implantable medical device comprising a structural material and an active agent, said coating dissolving when brought in contact with a cell and thereby allowing the release of said active agent wherein the dissolution is induced by the interaction between said cell and a substance contained in the structural material.

In one embodiment, the present invention relates to a coating for a medical device comprising a structural material, an active agent and a substance which is a temporary link between two structural materials to form the structure of the coating and/or between the active agent and the coating; wherein said structure material and/or said substance keep the active agent into the coating; wherein when brought into contact with a cell, said substance is adapted to release said active agent.

In one embodiment, the present invention relates to a coating for a medical device comprising a structural material, an active agent and a substance, the structural material and the substance forming together a structure of the coating in which the active agent is kept, wherein when brought into contact with a cell, the dissolution of the structure of the coating is induced allowing the release of said active agent.

In one embodiment, the present invention relates to a coating for an medical device comprising a structural material, an active agent and a substance, wherein the structural material and the substance form the structure of the coating in which the active agent is kept, wherein said substance is adapted to induce a dissolution of at least a part of said structure when the coating is brought into contact with or in presence of a specific cell or a specific state of a cell in such a way the active agent is released in a controlled manner.

In one embodiment, the present invention relates to a coating wherein the active agent, which may be a drug, is acting on the cell that initiated the dissolution.

In other words, the present invention relates to a coating for a medical device characterized in that:
the coating dissolves when put in contact with living cells, and/or
the dissolution of the coating is initiated by the cells, and/or
the coating contains an active agent which is released consequently to this dissolution, and/or
the coating prevents the release of the active agent until the cells are brought in contact with the coating.

In a preferred embodiment, this invention relates to the creation of a layer that can be coated onto an implant and that will react and release the active agent it contains in the presence, and only in the presence, of cells. This implant may be a stable implant that will remain in place after the dissolution of the coating. It may also be made out of degradable material and be resorbed over time after the dissolution of the coating. The coating itself may be made of different sub-coatings made of different materials. Sub-coating may differ by their structural material, their active agent and/or their substance inducing dissolution. These different sub-coatings may be arranged in different layers. A first layer may have to be completely dissolved to allow the beginning of the dissolution of the second layer. In another embodiment, the different sub-coatings may be located on different areas of the substrate. They may be in contact with each other or separated by a non-coated region. Their position on the substrate may be defined randomly or may be adjusted to present different responses according to the type of surrounding environment. Some regions of the substrate may be coated with a first type of coating while some other regions may be coated with a second type of coating having different properties.

Coatings offer the advantage of modifying the surface properties of implantable devices while keeping unchanged most of the substrate properties such as for example its mechanical behaviour. In a possible embodiment the coating is dimensioned in order to be fully dissolved when the action of the active agent (in particular a drug) isn't needed anymore. This is of particular interest, as a common problem with polymeric coating is their very poor long term biocompatibility. The release of small particles or chemical by-products (resulting in particular from the ageing of polymers) has been often described as a possible origin of toxicity or carcinogenic effects. The use of a material that disappears completely in the hours, days, weeks or months following the implantation should completely cancel this risk.

The coating in this invention contains an active agent. This agent is intended to be active after the implantation of the device and act on the surrounding cells or tissue. Contrary to other drug eluting coatings described extensively in the literature, it is supposed to be activated by cells present in the environment or cells present into the surrounding tissue and in a preferred embodiment by the targeted cells themselves or by the cells present in the targeted tissue. In a possible embodiment, the active agent will only be released after the coating has been put in contact with a particular tissue, with specific cells.

In a preferred embodiment, the dissolution of the coating will be initiated by defined and limited cell types. Ideally only a single cell type shall be able to initiate the degradation of a given coating. In that sense the coating is specific to a given cell-type. In a possible approach, the coating will react with an enzyme synthetized by, or present at the surface of a specific cell (which may be additionally the target of the drug contained in the coating). In other words, the coating will be made of an enzyme-sensitive biomaterial. This approach offers several advantages. First, due to the remarkable substrate selectivity of enzymes and the large database of identified enzyme, a specific coating can potentially be developed for each cell type. Then, as the expression and activity of enzymes may, for a given cell, be closely linked with their general condition (a cell in an inflamed or diseased tissue will express different enzymes than the same cell under normal conditions), it becomes possible to finely tailor the response of the coating to the surrounding tissue: a given cell-type will activate the coating only when certain physiological conditions are met.

In a preferred embodiment, the dissolution of the coating will only take place in presence of the target cells. If for any reason the target cells aren't anymore in contact with the tissue, the dissolution process will stop. Some active agents loaded in coatings may sometime be very toxic. They are meant for a given cell-type but are also often detrimental to other cells that may be present in the surroundings. It is therefore preferable to only release the active agent when needed, i.e. in the presence of the targeted tissue. In that sense the coating becomes really specific and minimizes side-effects as it releases its content in the presence of the targeted cells and only in their presence.

The active agent loaded into the coating may have different functionalities. In the case of a coating for drug eluting stents, it may be decided to use a toxic agent. This may be a drug such as for example paclitaxel, sirolimus, everolimus, tacrolimus, zotarolimus or biolimus, agents which are commonly used today. For orthopaedic implants, where a recurrent problem is post-operative infection, the drug may be chosen in the group of bacteriostatic or antibiotic compounds. In some other applications, the drug to be released may block the target cell to initiate a reaction chain. In the case of a peritonitis, the macrophages when moving to the bacteria colony will initiate a cascade reaction that will result in the creation of new blood vessel to increase the irrigation of the peritoneal membrane. This has a long term detrimental effect on the capacity of the membrane to act as a filtration and purification system. A inhibition of this cascade will protect the integrity of the membrane on the long run. In other applications, where an accelerated growth of the surrounding tissue may be desirable, agents favouring cell proliferation may be selected. Another non limiting example is the integration of a contrast medium in the coating. This compound will allow a non-invasive analysis of the tissue reaction after implantation. Imaging of the surrounding tissue may also be obtained using a molecule reactive to visible and non-visible light.

In another embodiment, the active agent loaded into the coating may be composed of different molecules. These may be different molecules with the same functionality, such as different bacteriostatic elements offering a broader activity spectrum. This may also be molecules having different functionalities: a combination of a cytotoxic drug and a contrast medium, for example. In that case it would become possible to diagnose and image a reaction of the surrounding tissue and treat it at the same time.

It is interesting to note here the potency of this approach in the case of bacterial infection. With this type of approach, it is possible to design a coating that will react and only react to a specific type of bacteria. An active agent such as an antibiotic that will be loaded in the coating will be especially targeted toward these bacteria and chosen for its efficacy. In the presence of the bacteria, it will be released locally with high efficiency. The antibiotic will however not be released if the targeted bacteria is not present and this will therefore limit the risk of generating resistant strains. This is only an example and the same approach may be applied to other diseases and therapies.

Another example of the potency of this approach is the possibility to give an advanced response to a complex cellular process. For example, the vascular injury following the placement of a stent in an artery will result in a series of events that can roughly be categorized into thrombosis, inflammation, proliferation of the SMC in the intima and media of the artery, migration of the medial SMC into the intima, and secretion of an extracellular matrix. By placing at the surface of the stent different coatings reacting to the presence of different cells (or to the same cell but under different physiological conditions), it becomes possible to release different drugs depending of the event currently occurring and encouraging or, on the contrary, inhibiting such events.

This coating also allows a temporal and spatial control of the drug release. This release is additionally initiated and controlled by the targeted cells. It therefore allows a smart and efficient answer with limited secondary effects.

The coating may be made out of various materials but in a preferred embodiment it will be made of a polymeric material, and more precisely of a hydrogel. Such a typical coating will contain at least two elements: a polymer structure and cell-sensitive moieties. The polymer will ideally be non-soluble and biocompatible. For example it may be made of multi-branched PEG chains or dendrimer molecules. Multi-branching is quite favourable to the creation of the mesh-type, porous structure of the coating. The cell-sensitive moiety will ideally be an enzyme-sensitive entity. In the absence of the targeted cells, the coating is stable. The size of the porosity is, in a preferred embodiment, chosen so that active agent molecules cannot diffuse into the surrounding tissue. In the presence of the target cell, the cell-sensitive moiety will be cleaved, destroying the mesh structure of the coating and liberating the active compound.

In a preferred embodiment, the spacer will be made of a series of peptides. These peptides will be cleaved by enzymes secreted or present at the surface of the target cells. The combinations peptide-enzyme is very potent due to its diversity and specificity. The literature gives a lot of examples of peptide sequences enzyme couples. Typical enzymes that have been studied are for example azoreductase, tyrosine kinase, cathepsins, alkaline and acid phosphatase, matrix metalloproteinases, elastase, lipase, 33-lactamase, thermolysin, plasmin, collagenase, urokinase, plasminogen-activator or factor Xa.

In some cases it may be of interest to additionally link the active agent to the matrix of the coating and in particular to the polymer part of the coating. This may be the case for small molecules that could be smaller than the size of the porosity of the coating. If these molecules were not attached, they could be released simply by diffusion through the structure. The link between the active agent and the coating may be soluble or stable. A soluble link will be preferred for ingredient that may lose their activity (or see it dramatically reduced) if linked to a large molecule. In a preferred embodiment, this soluble link is also specifically broken in the presence of the activating cell or of the target cell. In one embodiment, this link may be sensitive to the same triggering element than the one acting on the cell-sensitive moiety while in another embodiment it may be sensitive to another element.

The table below gives different possible approach as a function of the relatives sizes of the active ingredient, the activating molecule such as for example the enzyme expressed by the cell and the porosity of the coating. When we talk about the size of these different elements, we have to consider it as the size of these elements and what is “attached” to it. An given enzyme may have a size that would allow it to pass through the mesh of a given coating, but if it is always present at the surface of a cell, it has to be considered as an enzyme larger than the mesh size. In the same way, if an active ingredient when linked for example to a particle becomes larger than the mesh size of the coating, it has to be considered as being larger than the mesh size.

| | Enzyme size smaller than mesh size | Enzyme size larger than mesh size |
|---|---|---|
| Active agent | Active agent shall be linked to the polymeric | Active agent shall be linked to the polymeric |
| size smaller | material. This link may be non-soluble if the | material. This link may be non-soluble if the active |
| than mesh | active agent remains active when combined with | agent remains active when combined with the |
| size | the polymer or should be soluble otherwise. The | polymer or should be soluble otherwise. In |
| | dissolution of the coating being bulk dissolution, | particular it may ideally be cleaved by the enzyme |
| | the relative speed of the cleavage of the two | cleaving the coating itself. The dissolution of the |
| | links (active agent and coating) has to be | coating will be surface dissolution. |
| | chosen carefully. | |
| Active agent | Active agent may simply be entrapped into the | Active agent may simply be entrapped into the |
| size larger | coating structure. Dissolution of the coating will | coating structure. Dissolution of the coating will be |
| than mesh | be bulk dissolution. | surface dissolution. |
| size | | |

In a possible embodiment, the soluble link between the active agent and the coating may be an enzyme reactive molecule. In the case of a coating where dissolution is initiated by an enzyme, the link may react to the same enzyme or it may react to a different enzyme. In the case where the enzyme used to dissolve the coating differs for the enzyme used to detach the drug molecule, both reaction may be induced by the same cell (in which case this cell expresses both enzymes) or by distinct cells.

Here again the active agent present in the coating doesn't have to be limited to a single molecule. Several different molecules having similar or very different effect on the surrounding tissue may be linked to the coating through a soluble or non-soluble link. As above, the parameters driving the dissolution of the coating and the potential cleavage of the link between the active agent and the coating have to be chosen according to the specific application.

In a possible embodiment, the active agent may be independent from the coating, i.e. not linked to it, but it may be connected to another structure: e.g. polymeric chain, nano or micro particle, targeting moiety. This other structure may be passive or have an activity.

As mentioned in the table above, if the size of the active agent is larger than the size of the porosity of the coating, the active agent may simply be entrapped into the coating structure. It may be dissolved or precipitated into the coating. In may also, in another embodiment, be further encapsulated and the capsules may themselves be entrapped into the coating. The material of these capsules may further be degradable. It may also be attached to a carrier: polymer chains, branched polymer chains, dendrimers, particles . . . .

A known drawback of coated materials is the weakness of the mechanical interface between the substrate and the coating itself. Most of the time, coating and substrate materials are different and adhesion is purely mechanical. The roughness of the substrate acts as an anchoring structure to which the coating will adhere. The presence of a chemical link between the coating and the substrate (in addition to the mechanical link) will increase the quality of the interface and improve its resistance to wear, tear or fatigue. It may therefore be preferable in some embodiments to use an adapted chemistry to create (in addition to a mechanical link) a chemical link between the substrate and the coating. This chemical link may take the form of an intermediary layer such as poly-(L-lysine)-g-poly-(ethylene glycol) (PLL-g-PEG) grafted co-polymers.

### FIGURES

FIG. 1 shows a first possible embodiment of the invention where the target cell cleaves the peptide links to dissolve the coating and, as a consequence, liberate the active ingredient.

FIG. 2 shows another possible embodiment of the innovation where the target cell first cleaves the peptide links (dissolving the coating) to access the active agent and then cleaves the link between the active agent and the coating.

FIG. 3 shows a part of the cascade of reactions that take place during a peritonitis

### DETAILED DESCRIPTION OF THE INVENTION

In the following detailed description, reference is made to the accompanying drawings that form a part hereof, and in which are shown by way of illustration several embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, any direction referred to herein, such as “top”, “bottom”, “left”, “right”, “upper”, “lower”, and other directions or orientations are described herein for clarity in reference to the figures and are not intended to be limiting of an actual device or system. Devices and systems described herein may be used in a number of directions and orientations.

As used herein, “have”, “having”, “include”, “including”, “comprise”, “comprising” or the like are used in their open ended sense, and generally mean “including, but not limited to.

As used in this specification and the appended claims, the term “or” is generally employed in its sense including “and/or” unless the content clearly dictates otherwise.

According to an embodiment of this invention, a coating is provided for an implantable medical device wherein the coating is made of a structural material and a substance that, in the presence of a certain or several types of cells, will initiate its dissolution. In addition, the coating contains an active agent, dispersed into its structure or covalently linked to it, that will be liberated as a consequence of its dissolution.

Examples of medical devices benefiting from the present invention vary widely and include implantable (by implantable we mean implantable or insertable) medical devices, for example, catheters (e.g., urological catheters or vascular catheters such as balloon catheters and various central venous catheters), guide wires, balloons, filters (e.g., vena cava filters and mesh filters for distil protection devices), stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, embolization devices including cerebral aneurysm filler coils (including Guglilmi detachable coils and metal coils), septal defect closure devices, myocardial plugs, patches, pacemakers, lead coatings including coatings for pacemaker leads, defibrillation leads, and coils, ventricular assist devices including left ventricular assist hearts and pumps, total artificial hearts, shunts, valves including heart valves and vascular valves, anastomosis clips and rings, cochlear implants, active, programmable or passive pumps, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, joint prostheses, orthopedic fixation devices such as interference screws in the ankle, knee, and hand areas, tacks for ligament attachment and meniscal repair, rods and pins for fracture fixation, screws and plates for craniomaxillofacial repair, dental implants, or other devices that are implanted or inserted into the body and from which therapeutic agent is released or accessed.

Thus, while the devices on which the coating of the invention is applied may, in some embodiments, simply provide a substrate for controlled release of one or more therapeutic agents as a dosage form, in other embodiments, the medical devices on which the coating of the invention is applied are configured to provide a therapeutic function beyond targeted response to the environment, for instance, providing mechanical, thermal, pharmacological, magnetic and/or electrical functions within the body, among other many possible functions.

The medical devices of the present invention include, for example, implantable and insertable medical devices that are used for systemic treatment, as well as those that are used for the localized treatment of any mammalian tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth, adrenal gland, appendix, gall bladder, mouth, nose, parathyroid gland, pineal gland, pituitary gland, spleen, thymus, thyroid gland, vermiform appendix and bone.

As used herein, “treatment” refers to the prevention of a disease or condition, the reduction or elimination of symptoms associated with a disease or condition, or the substantial or complete elimination of a disease or condition. Preferred subjects are vertebrate subjects, more preferably mammalian subjects and more preferably human subjects.

Substrate materials for the medical devices on which the coating of the present invention is applied may vary widely in composition and are not limited to any particular material. They can be selected from a range of biostable materials and biodisintegrable, also mentioned in this text as biodegradable, materials (i.e., materials that are dissolved, degraded, resorbed, or otherwise eliminated upon placement in the body), including (a) organic materials (i.e., materials containing organic species, typically 50 wt. % or more) such as polymeric materials (i.e., materials containing polymers, typically 50 wt. % or more polymers) and biologics, (b) inorganic materials (i.e., materials containing inorganic species, typically 50 wt. % or more), such as metallic materials (i.e., materials containing metals, typically 50 wt. % or more) and non-metallic inorganic materials (e.g., including carbon, semiconductors, glasses and ceramics, which may contain various metal- and non-metaloxides, various metal- and non-metal-nitrides, various metal and non-metal-carbides, various metal- and non-metal-borides, various metal- and non-metal-phosphates, and various metal- and non-metal-sulfides, among others), and (c) hybrid materials (e.g., hybrid organic-inorganic materials, for instance, polymer/metallic inorganic and polymer/non-metallic inorganic hybrids).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, iron, niobium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic inorganic materials may be selected, for example, from metals such as gold, iron, niobium, platinum, palladium, iridium, osmium, rhodium, titanium, tantalum, tungsten, ruthenium, zirconium, and magnesium, among others, and alloys such as those comprising iron and chromium (e.g., stainless steels, including platinum-enriched radiopaque stainless steel), alloys comprising nickel and titanium (e.g., Nitinol) or other shape memory alloys, titanium alloys (e.g. Ti-6Al-4V), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), and biodisintegrable alloys including alloys of magnesium and/or iron (and their alloys with combinations of Ce, Ca, Zn, Zr and Li), among others.

Specific examples of organic materials include polymers (biostable or biodisintegrable/biodegradable) and other high molecular weight organic materials, and may be selected, for example, from suitable materials containing one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), poly-(ethylene glycol) (PEG), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kratone®G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-, l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, and glycosaminoglycans such as hyaluronic acid; as well as blends and further copolymers of the above.

For the sake of simplicity and of clarity, in the following description of the invention we will concentrate on coatings made of a polymeric substance, where the dissolution is induced using an enzymatic reaction. This invention however encompasses coatings made out of other materials such as for example those described above for the device, where the dissolution may be initiated by cell induced non-enzymatic reactions.

Under normal conditions, enzymes are tightly regulated to coordinate biochemical and biophysical activities necessary to sustain life. Enzyme expression is often related to cell activity. Enzymes and diseases are closely associated therefore the expression and activity of enzymes are good indicators of the progression and prognosis of a disease. Linking the activity of a coating to the expression of an enzyme is therefore very interesting. Their multitude (several thousands of enzymes have been identified to date), their very high specificity towards a predefined substrate and their link to different physiological states of a living tissue make them very interesting candidates as activators of smart drug eluting coatings.

For example, in the case of drug eluting stents, the smooth muscles cells present after the stenting express large amounts of MMP-2 and MMP-9 proteases. Johnson and Gallis (in C. Johnson and Z. Galis, “Matrix Metalloproteinase-2 and -9 Differentially Regulate Smooth Muscle Cell Migration and Cell-Mediated Collagen Organization”, Arterioscler. Thromb. Vasc. Biol., 24 (2004) 54-60) have demonstrated that hyperplasia conducting to restenosis which is observed after stenting could be strongly reduced in mice where the genes governing the synthesis of MMP-2 and MMP-9 has been knocked out. On the contrary, an over-expression of MMP-9 in the carotid of a rat model generated a higher migration of SMC and a more intense remodelling of the tissue. By choosing a material sensitive to MMP-2 or MMP-9, it becomes possible to specifically act in the presence of SMC involved in the restenosis process. A coating liberating at this point of time a toxic substance against these cells would prevent them to proliferate anarchically and, as a consequence, block the lumen of the artery.

A typical coating described in this invention is made up of three elements:
a polymer structure,
an enzyme sensitive cross-linking agent placed between the chains of the polymer and
active molecules such as for example drug.

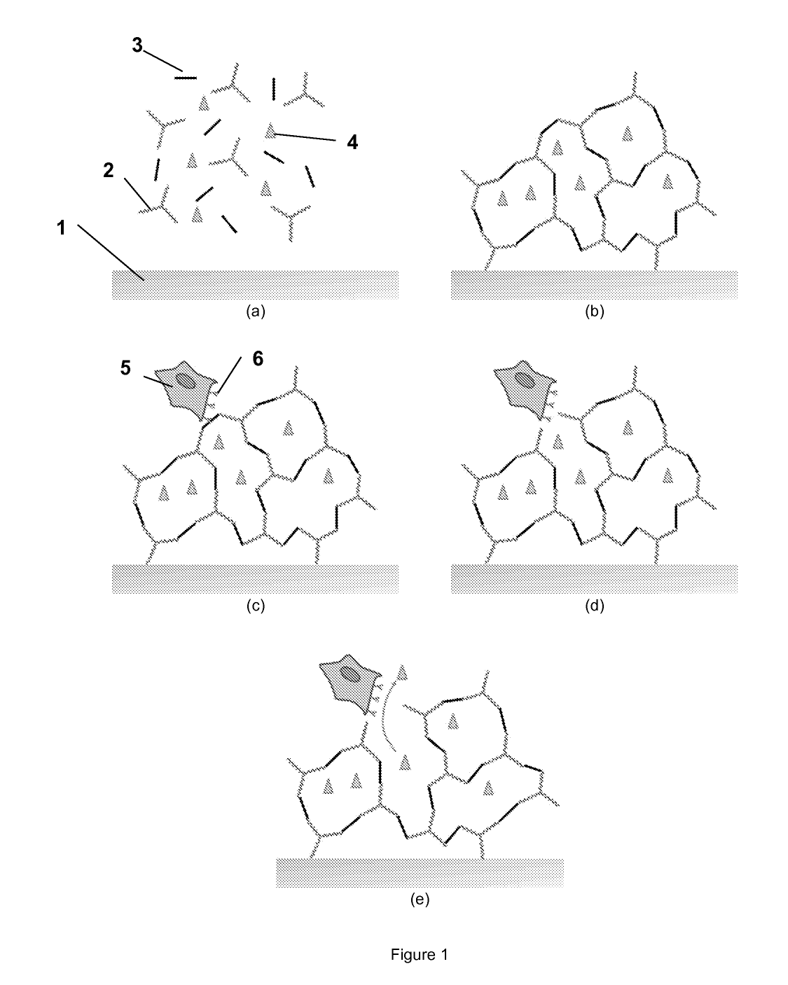

The FIG. 1(a) shows these different elements before there combination to create a coating: multi-branched polymer chains (2), peptide cross-linkers (3) and drug molecules (4). In the drawing, the represented polymer has a simple, relatively homogeneous structure. In another possible embodiment, the polymer may be less regular. Branching may occur, for example, along a long backbone. In another possible embodiment, the branching may be very high. The polymeric unit may be a dendrimer. On a surface (see FIG. 1(b)) the multi-branched polymer chains (2) and the peptide cross-linkers are combined to form a three dimensional network. The linked between the surface and the coating may be chemical as suggested by the drawing, but it may also be mechanical or a mix of both. During the formation process, the drug molecules (4) are trapped into the network. The two steps of surface coating and coating formation may occur at the same time or sequentially. For example, in the case of a polymeric coating, the polymerization (and as a consequence the drug entrapment for free molecules) may occur at the time of the coating deposition or it may happen before the coating deposition on the substrate. In the presence of a cell (5) (FIG. 1(c)) expressing a predetermined enzyme (6) (in the picture the enzyme is represented as being attached to the cell surface, but this may also be an enzyme released by the cell or whose presence is due to the presence of the cell) peptide cross-linkers will be cleaved (FIG. 1(d)) creating openings into the network. This will allow the entrapped drug molecules (4) to be liberated (FIG. 1(e)). This process will continue as long as cells expressing the enzyme cleaving the cross-linking peptide will be present. With time, polymer chains will be liberated and cleared by the environment; the coating will be dissolved and the drug eluted.

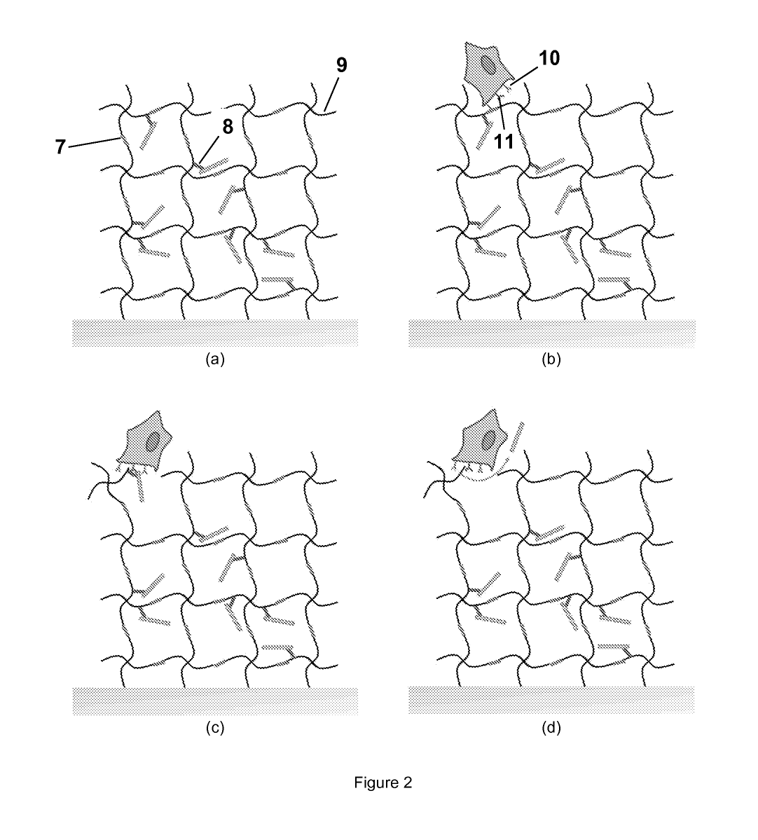

FIG. 2 shows another possible embodiment of the invention. Here again, the multi-branched polymer chains (9) are linked together by peptide cross-linkers (7). In this construction of the coating, drug molecules are attached to the coating through another peptide cross-linker than the one used to link the multi-branched polymer chains. These two peptide chains may react to enzyme present on or released by the same cell (as in the example shown in FIG. 2) or they may react to enzyme present on or released by different cells. In the case described in FIG. 2, the cell will first cleaves the peptide used to create the network (FIG. 2(b)) and then, after opening of the network, access to the drug molecule (FIG. 2(c)) and cleaves its link to the polymer chain. Again, as mentioned above, the enzyme may be attached directly to the cell, may be released by the cell or may have its presence being induced by the presence of the cell. For the sake of clarity and simplicity of this description, we talk about enzymatic reaction inducing both coating dissolution and drug release. Both are fully independent. Both, only one or none may be enzymatic reaction while the other one(s) will be cell induced non-enzimatic reactions. In this way it will liberate the molecule (FIG. 2(d)). In the case of peptides reacting to two different types of cells, the first type will destroy the network, inducing its dissolution, while the other will liberate the drug molecules.

### PEG-Based Coatings

Taking poly-(ethylene glycol) (PEG) as an example of basis material that could be used to create such coatings, there are two common methods of forming PEG-based hydrogels: chain growth and step growth polymerization of multifunctional PEG monomers.

In chain growth, PEG di(meth)acrylate monomers (PEGDMA) are polymerized by free radical chain polymerization, forming coiled poly(methacrylate) chains connected by PEG linkers. The advantage of this approach where the reaction is photoinitiated is to allow the formation of a hydrogel under physiological and cytocompatible conditions. The resulting molecular mesh size of the network is dictated by the length of the PEG chain and the concentration of PEGDMA in the gel-forming solution. In addition, the crosslinking density of the network is controlled by the concentration of PEGDMA, dictating the resultant gel modulus, equilibrium water content, and solute diffusivity. Degradation is easily introduced within these gels by incorporation of a degradable moiety within the PEGDMA crosslinking monomer.

In step growth polymerization, multifunctional PEG monomers are reacted stoichiometrically with degradable linkers to form nearly perfect networks by step growth polymerization. The reactive end groups may include acrylate and thiols polymerized by base-catalysed Michael-type reaction, norbornenes and thiols by free radical initiation, and azide and alkynes by copper-based or copper-free click reaction. Here the mesh size is dictated by the length of the PEG chain, whereas the crosslinking density is dictated by the length and concentration of the PEG crosslinker.

A possible way to create a coating as described in this invention is to use on one side multibranched PEG chains functionalized with vinyl sulfone groups and on the other side Cys-oligopeptides. A tri-dimensional network will be formed via a conjugate addition reaction (called Michael type reaction). This network will have plastic properties defined by the type of the used PEG precursor (in particular the length of the chains) as well as by the conditions of gel formation (in particular the pH at which the reaction takes places). The active agent can be added to the precursor solution and thus be entrapped into the final network.

The table below gives non limitative examples of known enzyme degradable sequence combinations. A major advantage of such sort of pairs is their high specificity.

| Enzyme | Degradable sequence |
|---|---|
| Lysosomal protease | GFLG |
| Collagenase | GGLGPAGGK |
| Elastase | AAAAAAAAAK; AAPVR; |
| | AAP(Nva) |
| Plasmin | VRN |
| Thrombin | G d-F Pipecolic |
| | Acid RSGGGGKC |
| Collagenase and various | GPQGIWGQ; GPQGIAGQ; |
| matrix metalloproteinases | GPQGILGQ |
| (MMPs), such as MMP-1, | |
| -2, -3, -7, -8 and -9 | |
| MMP-2 and MMP-9 | PVGLIG; GPAGLGC |
| MMP-2 | GPLGIAGQ |

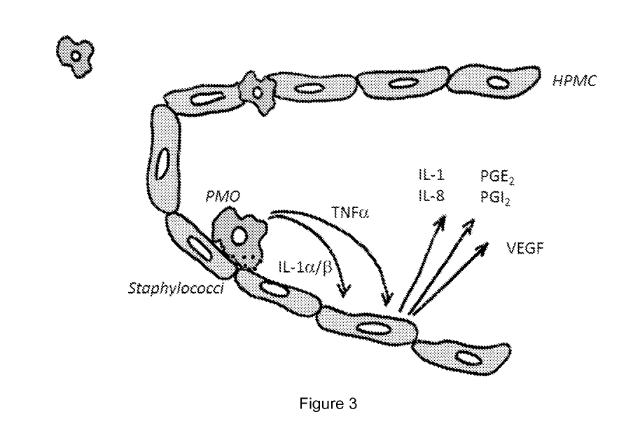

In a possible embodiment, the drug released by the coating is used to stop a signalling cascade and therefore block a reaction of the body to an external event. For example, in the peritoneal cavity, the presence of bacteria, such as for example staphylococci, may generate an infection, a peritonitis. Macrophages (PMO) will move to the bacteria colony and initiate its elimination through lysis (see FIG. 3). In parallel they will liberate various factors such as IL-1α/β or TNFα that will activate the mesothelial cells (HPMC) forming the peritoneal membrane. These HPMC will, in their turn, liberate further factors to 1) generate an inflation and recruit white blood cell to fight the infection (IL-6, IL-8, PGE₂, PGI₂) and 2) favour vascularization of the peritoneal membrane (VGEF). After the disappearance of the infection, the newly created vessels will remain, increasing the blood flow into the peritoneal membrane. This effect, in itself, is not an issue. It will become a problem for patients under peritoneal dialysis. Increasing the vascularization of their peritoneal membrane will increase the transport capacity of this membrane and as a consequence will make the therapy less efficient. In this example, the catheter which is implanted in the peritoneal cavity of the patient may be covered of a coating activated in the presence of VGEF and that will liberate a chemical entity that will block the activity of the same VGEF. In that way the cascade leading to the recruitment of white blood cells to fight infection is maintained while that leading to the vascularization of the membrane is blocked.

1. A coating, for a medical device, comprising an active agent which is released in a controlled manner, said coating furthermore comprising a structural material and substance, wherein the substance is adapted to induce the dissolution of the coating in such a way to release the active agent when said substance is in contact with or in presence of a specific cell.

2. A coating according to claim 1 wherein said cell is in a specific state.

3. A coating according to wherein the active agent is adapted to target a cell type.

4. A coating according to claim 3 wherein the dissolution of the coating takes place only in the presence of the cell type targeted by the active agent.

5. A coating according to claim 1 wherein the active agent is:
a toxic agent for the cells, which may be a compound of the list: paclitaxel, sirolimus, everolimus, tacrolimus, zotarolimus or biolimus; or
a contrast medium; or
an agent that favours cell proliferation.

6. A coating according to claim 1 wherein the active agent is composed of different molecules.

7. A coating according to claim 1 wherein the structural material is made of a polymeric material.

8. A coating according to claim 7 wherein the polymeric material is a hydrogel.

9. A coating according to claim 1 wherein the structural material is made of a combination of polymer coupled to peptides that are cleaved by enzymes released by cells or present at the surface of the cells.

10. A coating according to claim 9 wherein the enzymes are azoreductase, tyrosine kinase, cathepsins, alkaline and acid phosphatase, matrix metalloproteinases, elastase, lipase, -lactamase, thermolysin, plasmin, collagenase, urokinase, plasminogen-activator or factor Xa.

11. A coating according to claim 1 wherein the active agent is covalently attached to the coating.

12. A coating according to claim 1 wherein the active agent is coupled to the coating using a peptide.

13. A coating according to claim 1 wherein different molecules are coupled to the coating with different peptides.

14. A coating according to claim 1 wherein the active agent is dispersed within the coating material.

15. A coating according to claim 14 wherein the active agent dispersed into the matrix is first encapsulated into a degradable structure or attached to a polymer chain, a branched polymer, a dendrimer, or a particle

16. A coating according to claim 1 wherein an intermediate layer is created between the coating and the substrate to improve the adhesion of the coating onto the substrate.
